(19) United States
(12) Patent Application Publication
(10) Pub. No.: US 2003/0224075 A1
(43) Pub. Date: Dec. 4, 2003

(54) Soy composition for balancing combination skin

(76) Inventors: Jue-Chen Liu, Belle Mead, NJ (US); Jeffrey Wu, Warrington, PA (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/370,680

(22) Filed: Feb. 20, 2003

Related U.S. Application Data
(60) Provisional application No. 60/359,060, filed on Feb. 21, 2002.

(51) Int. Cl. A61K 7/06; A61K 35/78

(52) U.S. Cl. 424/757; 424/74

(57) ABSTRACT

The present invention features legume products, compositions containing such legume products, and the topical application of such legume products for treating combination skin and maintaining balanced facial skin.

### FIELD OF THE INVENTION

This invention relates to compositions and methods of treating combination skin so as to provide the benefit of balancing the skin's oily and dry areas such that the oily areas appear less shiny and the dry areas appear and feel more moist.

### BACKGROUND OF THE INVENTION

A large proportion of the adult population considers their facial skin to be a combination type with both dry and oily areas. There is a lack of effective skin care products on the market today that address this need.

Combination skin, defined as facial skin having areas of dryness as well as areas that are perceived to be overly oily, has presented great dilemmas in terms of treatment and appearance. Generally, individuals having combination skin attempt to treat areas of oiliness in order to prevent outbreaks of acne and to diminish shininess. However, such treatments and preparations tend to dry the skin and cause irritation to the areas of the skin that are dry. Similarly, if individuals attempt to treat dry patches of skin by applying moisturizing compositions, which generally contain large proportions of emollients and oils, such compositions tend to clog pores and increase oiliness in already-oily areas and generate comedone production. This type of treatment results in undesirable skin breakout or acne. Applying separate compositions to different areas of the face and neck is time-consuming, expensive and inconvenient

Thus, it would be desirable to have compositions and methods of treatment that address the condition of combination skin so as to permit treatment of combination skin with one composition. It would be further desirable that such a composition would balance the skin condition, in order to render the appearance of the skin neither overly-oily nor overly-dry.

### SUMMARY OF THE INVENTION

This invention relates to compositions and methods of selectively balancing the oiliness and dryness of human skin. By applying compositions containing non-denatured legume products, a user having “combination skin” (i.e., skin having areas of oiliness and areas of dryness in proximity) may obtain skin in which the oily areas produce less sebum and “oil” and the dry areas become moisturized. Topical soy-containing preparations have been shown to deliver significant skin care benefits. Such soy-containing preparations should preferably contain “total soy”, or soy products that contain non-denatured proteins. Such compositions are exemplified in copending U.S. patent application Ser. No. 09/796,054, filed Feb. 28, 2001 and U.S. patent application Ser. No. 09/795,762, filed Feb. 28, 2001, which are hereby incorporated herein by reference.

We believe that the components of “total soy” can help improve combination skin. In fact, total soy contains a balanced, complete spectrum of nutrients such as soy proteins, fatty acids, polysacchrides, vitamins, minerals, flavonoids and actives that provide multi-functional skincare benefits. We believe that other total legume-containing compositions may also confer the same type of benefit as those of non-denatured “total soy”.

Legume Product

What is meant by a “legume product” is a substance derived from a legume fruit. A legume is a plant from the family Leguminosae, which has a dehiscent fruit such as a bean, pea, or lentil. Examples of legumes, include but are not limited to, beans such as soybeans, lentil beans, peas, and peanuts.

The legume product may contain the entire legume fruit (e.g., the legume fruit ground into a powder) or only a portion of the legume (e.g., an extract of the legume). The legume product may be in the form of a fluid (e.g., a mixture of the legume fruit and water) or a solid (e.g., legume fruits powders). When in the form of a fluid, the term “legume product” refers to the solid constituents of the fluid derived from the legume.

The compositions of the present invention comprise a safe and effective amount of the legume product (e.g., soy product). In one embodiment, the composition contains from about 0.001% to about 50%, more preferably from about 1% to about 30% or most preferably from about 2% to about 20%, by weight, of the legume product (e.g., a soy product).

Soy Product/“Total Soy”

What is meant by a “Soy Product” or “total soy”, is a substance derived from the soybean. The soy product may contain only a portion of the soybean (e.g., an extract of the soybean such as a lipid reduced soybean powder or filtered soymilk) or may contain the entire soybean (e.g., a ground powder of the legume). The soy product may be in the form of a fluid (e.g., soymilk) or a solid (e.g., a soybean powder or soymilk powder). When in the form of a fluid, the term “soy product” refers to the solid constituents of the fluid that are derived from the soybean.

In one embodiment, the soy product is soybean powder. Soybean powder may be made by grinding dry soybeans. In one embodiment, the soybean powder has a average particle size of less than about 150 micrometers and possibly less than about 10 micrometers. In one embodiment, the soybean powder has a moisture content of less than about 10%, more preferably, less than about 5%. In one embodiment, the soybean powder may be lyophilized, or freeze-dried.

In one embodiment, the soy product is soymilk or soymilk powder. Soymilk is a combination of solids derived from soybeans and water, the mixture of which has some or all of the insoluble constituents filtered off. Soymilk powder is evaporated soymilk, which in one embodiment, is in a lyophilized or spray-dried form. Procedures for manufacturing soymilk include, but are not limited to, the following three procedures. First, soymilk may be made by placing soybeans into water to allow them to absorb the water. The swelled beans are then ground and additional water is then added. The mixture may then filtered to remove any insoluble residue. Second, soymilk may also be prepared from soybean powder. Soybean powder is thoroughly mixed with water (e.g., for at least one hour), which may then be followed by a filtration process to remove insoluble residues. Third, soymilk can also be reconstituted from soymilk powder by adding water. In one embodiment, soymilk comprises from between about 1% to about 50%, by weight (e.g., from about 5% to about 20%, by weight) of solids from the soybean.

A “safe and effective amount” means an amount of compound or composition (e.g., the legume product) sufficient to induce a positive modification in the condition to be regulated or treated, but low enough to avoid serious side effects. The safe and effective amount of the compound or composition will vary with the particular condition being treated, the age and physical condition of the end user, the severity of the condition being treated/prevented, the duration of the treatment, the nature of other treatments, the specific compound or product/composition employed, the particular cosmetically-acceptable carrier utilized, and like factors.

As used herein, “topical application” means directly laying on or spreading on outer skin using, e.g., by use of the hands or an applicator such as a wipe, puff, roller, or spray.

As used herein, “cosmetically-acceptable” means that the product(s) or compound(s) which the term describes are suitable for use in contact with tissues (e.g., the skin) without undue toxicity, incompatibility, instability, irritation, allergic response, and the like. This term is not intended to limit the ingredient/product to which it describes for use solely as a cosmetic (e.g., the ingredient/product may be used as a pharmaceutical).

As used herein, “topical carrier” means one or more compatible solid or liquid filler diluents that are suitable for topical administration to a mammal. Examples of topical carriers include, but are not limited to, water, waxes, oils, emollients, emulsifiers, thickening agents, gelling agents, and mixtures thereof.

As used herein, “trypsin inhibitory activity” means the ability of the legume product at a concentration of 0.1% (w/w) to inhibit the activity of the protease trypsin, as measured by the assay set forth below in Example 2. In one embodiment, the legume products of the present invention have a trypsin inhibitory activity of at least about 15%. In a further embodiment, the legume products of the present invention have a trypsin inhibitory activity of at least about 25%, such as at least about 50%. However, achieving skin balancing in accordance with this invention does not necessarily require the presence of high trypsin inhibitory activity.

“Objectionable microbial content” means the amount of bacteria, fungi, and yeast present in the legume product that are harmful to humans, including but not limited to coliform, E. Coli, Salmonella, thermophilic spores, Bacillus, Enterococcus, Staphylococcus, fecal streptococcus, and those listed in “Disinfection, sterilization, and preservation” 4th edition, Seymour S. Block, pp. 887-888 (1991, Lea & Febiger, Malvern, Pa.).

As used herein, “topical application” means directly laying on or spreading on outer skin using, e.g., by use of the hands or an applicator such as a wipe, puff, roller, or spray.

As used herein, “cosmetically-acceptable” means that the product(s) or compound(s) which the term describes are suitable for use in contact with tissues (e.g., the skin) without undue toxicity, incompatibility, instability, irritation, allergic response, and the like.

As used herein, “topical carrier” means one or more compatible solid or liquid filler diluents that are suitable for topical administration to a mammal. Examples of topical carriers include, but are not limited to, water, waxes, oils, emollients, emulsifiers, thickening agents, gelling agents, and mixtures thereof.

As used herein, “safe and effective amount” means an amount of compound or composition (e.g., the legume product) sufficient to induce a positive modification in the condition to be regulated or treated, but low enough to avoid serious side effects. The safe and effective amount of the compound or composition will vary with the particular condition being treated, the age and physical condition of the end user, the severity of the condition being treated/prevented, the duration of the treatment, the nature of other treatments, the specific compound or product/composition employed, the particular cosmetically-acceptable carrier utilized, and like factors.

Examples of compositions suitable for use in the compositions and methods of this invention are described in copending U.S. patent applications Ser. No. 09/110,409, filed Jul. 6, 1998, Ser. No. 09/206,249, filed Dec. 7, 1998, Ser. No. 09/361,429, filed Jul. 27, 1999, Ser. No. 09/621,565, filed Jul. 21, 2000 and Ser. No. 09/698,454, filed Oct. 27, 2000, which are hereby incorporated herein by reference.

Additional sources of nutrients similar to those in “total soy” may be contained in the species belonging to the following plant families: Solanaceae (e.g., potato, tomato, tomatilla, and the like); Gramineae (e.g., rice, buckwheat, sorghum, wheat, barley, oats and the like); Cucurbitaceae (e.g., cucumbers, squash, gourd, luffa and the like); and, preferably, Leguminosae (e.g., beans, peas, lentils, peanuts, and the like).

The compounds which are active in the compositions and methods of this invention may be delivered topically by any means known to those of skill in the art. If the delivery parameters of the topically active pharmaceutical or cosmetic agent so require, the topically active compositions of this invention may preferably be further composed of a pharmaceutically or cosmetically acceptable vehicle capable of functioning as a delivery system to enable the penetration of the topically active agent into the skin.

One acceptable vehicle for topical delivery of some of the compositions of this invention, particularly proteins such as trypsin and STI, may contain liposomes. The liposomes are more preferably non-ionic and contain a) glycerol dilaurate (preferably in an amount of between about 5% and about 70% by weight); b) compounds having the steroid backbone found in cholesterol (preferably in an amount of between about 5% and about 45% by weight); and c) one or more fatty acid ethers having from about 12 to about 18 carbon atoms (preferably in an amount of between about 5% and about 70% by weight collectively), wherein the constituent compounds of the liposomes are preferably in a ratio of about 37.5:12.5:33.3:16.7. Liposomes comprised of glycerol dilaurate/cholesterol/polyoxyethylene-10-stearyl ether/polyoxyethylene-9-lauryl ether (GDL liposomes) are most preferred. Preferably the liposomes are present in an amount, based upon the total volume of the composition, of from about 10 mg/mL to about 100 mg/mL, and more preferably from about 20 mg/mL to about 50 mg/mL. A ratio of about 37.5:12.5:33.3:16.7 is most preferred. Suitable liposomes may preferably be prepared in accordance with the protocol set forth in Example 1, though other methods commonly used in the art are also acceptable. The above described composition may be prepared by combining the desired components in a suitable container and mixing them under ambient conditions in any conventional high shear mixing means well known in the art for non-ionic liposomes preparations, such as those disclosed in Niemiec et al., “Influence of Nonionic Liposomal Composition On Topical Delivery of Peptide Drugs Into Pilosebacious Units: An In Vivo Study Using the Hamster Ear Model,” 12 Pharm. Res. 1184-88 (1995) (“Niemiec”), which is incorporated by reference herein in its entirety. We have found that the presence of these liposomes in the compositions of this invention may enhance the depigmenting capabilities of some of the compositions of this invention.

Other preferable formulations may contain, for example, soybean milk or other liquid formulations derived directly from legumes or other suitable plant. For example, such a formulation may contain a large proportion of soybean milk, an emulsifier that maintains the physical stability of the soybean milk, and, optionally a chelating agent, preservatives, emollients, humectants and/or thickeners or gelling agents.

Oil-in-water emulsions, water-in-oil emulsions, solvent-based formulations and aqueous gels known to those of skill in the art may also be utilized as vehicles for the delivery of the compositions of this invention.

The source of active compound to be formulated will generally depend upon the particular form of the compound. Small organic molecules and peptidyl fragments can be chemically synthesized and provided in a pure form suitable for pharmaceutical/cosmetic usage. Products of natural extracts can be purified according to techniques known in the art. Recombinant sources of compounds are also available to those of ordinary skill in the art.

In alternative embodiments, the topically active pharmaceutical or cosmetic composition may be optionally combined with other ingredients such as moisturizers, cosmetic adjuvants, anti-oxidants, bleaching agents, tyrosinase inhibitors and other known depigmentation agents, surfactants, foaming agents, conditioners, humectants, fragrances, viscosifiers, buffering agents, preservatives, sunscreens and the like. The compositions of this invention may also contain active amounts of retinoids (i.e., compounds that bind to any members of the family of retinoid receptors), including, for example, tretinoin, retinol, esters of tretinoin and/or retinol and the like.

Liquid derivatives and natural extracts made directly from plants or botanical sources may be employed in the compositions of this invention in a concentration (w/v) from about 1 to about 99%. Fractions of natural extracts and naturally-derived active ingredients such as STI may have a different preferred range, from about 0.01% to about 20% and, more preferably, from about 1% to about 10% of the composition. Of course, mixtures of the active agents of this invention may be combined and used together in the same formulation, or in serial applications of different formulations.

Preferably, the compositions of this invention contain from about 20% to about 99% of extracts of botanical materials; and from about 1% to about 20% for fractions of natural extracts and naturally-derived active ingredients (such as protease inhibitors, e.g., soybean trypsin inhibitor or the like and mixtures of active ingredients) between one and two times daily for a period of time until the skin evidences a change to a more balanced appearance.

Thereafter, once the change in appearance has been achieved, a lower concentration and dose from about 10% to about 90% for liquid derivatives and extracts of botanical materials; and from about 0.01% to about 5% for fractions of natural extracts and naturally-derived protease inhibitors such as STI or mixtures thereof), of active ingredient may be applied on a less frequent time schedule, e.g., about once per day to about twice per week. The effects of the active agents of this invention are reversible, therefore, in order to maintain these effects, continuous application or administration should be performed. The invention illustratively disclosed herein suitably may be practiced in the absence of any component, ingredient, or step which is not specifically disclosed herein.

The compositions effective in the methods of this invention contain a safe and effective amount of non-denatured legume product, most preferably, a soy product. The topical compositions useful in the present invention involve formulations suitable for topical application to skin.

Anti-Microbial Treatment of Legume Product

The surface of legume fruits often contain high levels of microorganisms. Thus, prior to use by humans, the legume product needs to be treated to reduce or eliminate such microorganisms.

In one embodiment, the legume products of the present invention have a total microbial content of less than about 10,000 colony-forming units (“cfu”) per gram. In a further embodiment, the soy products of the present invention have a microbial content of less than about 1,000 cfu per gram (such as less than about 100 cfu per gram) of the legume product.

In one embodiment, the legume products of the present invention have a total objectionable microbial content of less than 300 cfu per gram such as less than 150 cfu per gram. In a further embodiment, the legume products of the present invention have an undetectable amount of any objectionable microbials for at least one gram (e.g., at least ten grams) of legume product.

In one embodiment, the legume product is exposed to gamma irradiation. In a further embodiment, the legume product is exposed to between about 2 to about 30 kGy of gamma irradiation, such as between about 5 and about 10 kGy of gamma irradiation. Applicants have unexpectedly found that such treatment reduces the microbial content of the legume product, while maintaining its biological activity (e.g., serine protease inhibitory activity). Applicants have also found that treatment of legume products with gamma irradiation maintains the cosmetic elegance of the legume product, such as maintained its natural colors and did not induce significant malodors.

Other anti-microbial processes that also maintain the protease inhibitory activity of the legume product that can be practiced alone or in combination with gamma irradiation, include, but are not limited to, exposure to x-rays, high energy electron or proton beams, ultraviolet radiation, hydrostatic pressure, and addition of chemical agents possessing antimicrobial activity, and combinations thereof. A complete list of methods for microbial content reduction is set forth in “Disinfection, sterilization, and preservation” 4th edition, Seymour S. Block, pp. 887-888 (1991, Lea & Febiger, Malvern, Pa.).

Applicants have found that processes using thermal treatment may result in a substantial loss in protease inhibitory activity and, thus, should be used with caution. For example, applicants have found that heating soymilk to 100° C. for only 10 minutes reduced the trypsin inhibitory activity of the soymilk from 86% (when maintained at 4° C.) to 46%. Applicants have found that heating soymilk can also result in a change of the color or odor of the soybean product.

Topical Compositions

The topical compositions useful in the present invention involve formulations suitable for topical application to skin. In one embodiment, the composition comprises the soy product and a cosmetically-acceptable topical carrier. In one embodiment, the cosmetically-acceptable topical carrier is from about 50% to abut 99.99%, by weight, of the composition (e.g., from about 80% to about 95%, by weight, of the composition.

The compositions may be made into a wide variety of product types that include but are not limited to lotions, creams, gels, sticks, sprays, shaving creams, ointments, cleansing liquid washes and solid bars, shampoos, pastes, powders, mousses, shaving creams, wipes, patches, nail lacquers, wound dressing and adhesive bandages, hydrogels, films and make-up such as foundations, mascaras, and lipsticks. These product types may comprise several types of cosmetically acceptable topical carriers including, but not limited to solutions, emulsions (e.g., microemulsions and nanoemulsions), gels, solids and liposomes. The following are non-limitative examples of such carriers. Other carriers can be formulated by those of ordinary skill in the art.

The topical compositions useful in the present invention can be formulated as solutions. Solutions typically include an aqueous solvent (e.g., from about 50% to about 99.99% or from about 90% to about 99% of a cosmetically acceptable aqueous solvent).

Topical compositions useful in the subject invention may be formulated as a solution comprising an emollient. Such compositions preferably contain from about 2% to about 50% of an emollient(s). As used herein, “emollients” refer to materials used for the prevention or relief of dryness, as well as for the protection of the skin. A wide variety of suitable emollients are known and may be used herein. Sagarin, Cosmetics, Science and Technology, 2nd Edition, Vol. 1, pp. 32-43 (1972) and the International Cosmetic Ingredient Dictionary and Handbook, eds. Wenninger and McEwen, pp. 1656-61, 1626, and 1654-55 (The Cosmetic, Toiletry, and Fragrance Assoc., Washington, D.C., 7^{th} Edition, 1997) (hereinafter “ICI Handbook”) contains numerous examples of suitable materials.

A lotion can be made from such a solution. Lotions typically comprise from about 1% to about 20% (e.g., from about 5% to about 10%) of an emollient(s) and from about 50% to about 90% (e.g., from about 60% to about 80%) of water.

Another type of product that may be formulated from a solution is a cream. A cream typically comprises from about 5% to about 50% (e.g., from about 10% to about 20%) of an emollient(s) and from about 45% to about 85% (e.g., from about 50% to about 75%) of water.

Yet another type of product that may be formulated from a solution is an ointment. An ointment may comprise a simple base of animal or vegetable oils or semi-solid hydrocarbons. An ointment may comprise from about 2% to about 10% of an emollient(s) plus from about 0.1% to about 2% of a thickening agent(s). A more complete disclosure of thickening agents or viscosity increasing agents useful herein can be found in Sagarin, Cosmetics, Science and Technology, 2nd Edition, Vol. 1, pp. 72-73 (1972) and the ICI Handbook pp. 1693-1697.

The topical compositions useful in the present invention formulated as emulsions. If the carrier is an emulsion, from about 1% to about 10% (e.g., from about 2% to about 5%) of the carrier comprises an emulsifier(s). Emulsifiers may be nonionic, anionic or cationic. Suitable emulsifiers are disclosed in, for example, U.S. Pat. Nos. 3,755,560, 4,421,769, McCutcheon's Detergents and Emulsifiers, North American Edition, pp. 317-324 (1986), and the ICI Handbook, pp.1673-1686.

Lotions and creams can be formulated as emulsions. Typically such lotions comprise from 0.5% to about 5% of an emulsifier(s). Such creams would typically comprise from about 1% to about 20% (e.g., from about 5% to about 10%) of an emollient(s); from about 20% to about 80% (e.g., from 30% to about 70%) of water; and from about 1% to about 10% (e.g., from about 2% to about 5%) of an emulsifier(s).

Single emulsion skin care preparations, such as lotions and creams, of the oil-in-water type and water-in-oil type are well-known in the cosmetic art and are useful in the subject invention. Multiphase emulsion compositions, such as the water-in-oil-in-water type, as disclosed in U.S. Pat. Nos. 4,254,105 and 4,960,764, are also useful in the subject invention. In general, such single or multiphase emulsions contain water, emollients, and emulsifiers as essential ingredients.

The topical compositions of this invention can also be formulated as a gel (e.g., an aqueous gel using a suitable gelling agent(s)). Suitable gelling agents for aqueous gels include, but are not limited to, natural gums, acrylic acid and acrylate polymers and copolymers, and cellulose derivatives (e.g., hydroxymethyl cellulose and hydroxypropyl cellulose). Suitable gelling agents for oils (such as mineral oil) include, but are not limited to, hydrogenated butylene/ethylene/styrene copolymer and hydrogenated ethylene/propylene/styrene copolymer. Such gels typically comprises between about 0.1% and 5%, by weight, of such gelling agents. The topical compositions of the present invention can also be formulated into a solid formulation (e.g., a wax-based stick, soap bar composition, powder, or a wipe containing powder).

The topical compositions useful in the subject invention may contain, in addition to the aforementioned components, a wide variety of additional oil-soluble materials and/or water-soluble materials conventionally used in compositions for use on skin, hair, and nails at their art-established levels.

Additional Cosmetically Active Agents

In one embodiment, the topical composition further comprises another cosmetically active agent in addition to the legume product. What is meant by a “cosmetically active agent” is a compound that has a cosmetic or therapeutic effect on the skin, hair, or nails, e.g., lightening agents, darkening agents such as self-tanning agents, anti-acne agents, shine control agents, anti-microbial agents, anti-inflammatory agents, anti-mycotic agents, anti-parasite agents, external analgesics, sunscreens, photoprotectors, antioxidants, keratolytic agents, detergents/surfactants, moisturizers, nutrients, vitamins, energy enhancers, anti-perspiration agents, astringents, deodorants, hair removers, firming agents, anti-callous agents, and agents for hair, nail, and/or skin conditioning.

In one embodiment, the agent is selected from, but not limited to, the group consisting of hydroxy acids, benzoyl peroxide, sulfur resorcinol, ascorbic acid, D-panthenol, hydroquinone, octyl methoxycinnimate, titanium dioxide, octyl salicylate, homosalate, avobenzone, polyphenolics, carotenoids, free radical scavengers, spin traps, retinoids such as retinol and retinyl palmitate, ceramides, polyunsaturated fatty acids, essential fatty acids, enzymes, enzyme inhibitors, minerals, hormones such as estrogens, steroids such as hydrocortisone, 2-dimethylaminoethanol, copper salts such as copper chloride, peptides containing copper such as Cu:Gly-His-Lys, coenzyme Q10, peptides such as those disclosed in PCT Patent Application WO00/15188, lipoic acid, amino acids such a proline and tyrosine, vitamins, lactobionic acid, acetyl-coenzyme A, niacin, riboflavin, thiamin, ribose, electron transporters such as NADH and FADH2, and other botanical extracts such as aloe vera, and derivatives and mixtures thereof. The cosmetically active agent will typically be present in the composition of the invention in an amount of from about 0.001% to about 20% by weight of the composition, e.g., about 0.01% to about 10% such as about 0.1% to about 5%.

Examples of vitamins include, but are not limited to, vitamin A, vitamin Bs such as vitamin B3, vitamin B5, and vitamin B12, vitamin C, vitamin K, and vitamin E and derivatives thereof.

Examples of hydroxy acids include, but are not limited, to glycolic acid, lactic acid, malic acid, salicylic acid, citric acid, and tartaric acid. See, e.g., European Patent Application No. 273,202.

Examples of antioxidants include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isoflavonoids and their derivatives (e.g., genistein and diadzein), extracts containing resveratrol and the like. Examples of such natural extracts include grape seed, green tea, pine bark, and propolis. Other examples of antioxidants may be found on pages 1612-13 of the ICI Handbook.

Other Materials

Various other materials may also be present in the compositions useful in the subject invention. These include humectants, proteins and polypeptides, preservatives and an alkaline agent. Examples of such agents are disclosed in the ICI Handbook, pp.1650-1667. The compositions of the present invention may also comprise chelating agents (e.g., EDTA) and preservatives (e.g., parabens). Examples of suitable preservatives and chelating agents are listed in pp. 1626 and 1654-55 of the ICI Handbook. In addition, the topical compositions useful herein can contain conventional cosmetic adjuvants, such as dyes, opacifiers (e.g., titanium dioxide), pigments, and fragrances.

Mineral Water

The legume product (e.g., soymilk) and compositions of the present invention may be prepared using a mineral water. In one embodiment, the mineral water has a mineralization of at least about 200 mg/L (e.g., from about 300 mg/L to about 1000 mg/L). In one embodiment, the mineral water comprises at least about 10 mg/L of calcium and/or at least about 5 mg/L of magnesium.

The composition and formulations containing such compositions of the present invention may be prepared using methodology that is well known by an artisan of ordinary skill.

### EXAMPLE 1

Gamma Irradiation of Legume Product

Applicants have found that soymilk powder prior to any antimicrobial processing such as gamma irradiation has high levels microbial content, ranging from up to 50,000 cfu per gram. Such products were also found to have detectable levels of objectionable microbial content, such as fecal streptococci, at levels up to 20,000 cfu per gram.

Applicants have exposed various amounts (e.g., from about 1 g to about 200 kg) of soymilk powder to gamma irradiation varying from 1 kGy to 16 kGy. The dose or gamma irradiation needed for a reduction a total microbial content to less than about 100 cfu per gram was found to be about 10 kGy. The dose for one log reduction for fecal streptococci is determined to be about 3 kGy and a dose of about 5 kGy was found to consistently reduce this microbial content within a 10 gram sample of soymilk powder to undetectable levels. However, the amount of gamma irradiation used on the legume product will ultimately be determined by the microbial content and size of the soy product to be so treated.

### EXAMPLE 2

Trypsin Inhibitory Activity of Legume Product

The inhibition of trypsin-induced cleavage of a fluorescent casein peptide was measured using the EnzChek™ protease assay kit, following manufacturer's instructions (EnzChek™ Protease Assay Kits Product Information, Revised Mar. 15, 1999; Molecular Probes, Eugene Oreg.). In summary, various soy preparations were first diluted in 1× digestion buffer (provided in kit) and incubated at different concentrations with 1000 units of trypsin (Sigma, St. Louis, Mo.) dissolved in 1× digestion buffer. A pure serine protease inhibitor (soybean trypsin inhibitor, from Sigma, St. Louis, Mo.) was used as a positive control at 0.1, 0.01%, and 0.001% w/v. Then, 1.0 mg/ml stock solution of BODIPY FL casein was prepared by adding 0.2 mL of deionized water to the vials supplied with this substrate (provided in kit), then made to a final working concentration of 10 microgram/ml in digestion buffer. Following incubation of the trypsin, with or without the test material, with the BODIPY fluorescent casein substrate at room temperature for one hour, fluorescence was measured (excitation 485 nm/emission 530 nm) on a SpectraMax® Gemini microtiter plate reader (Molecular Devices Corporation, Sunnyvale, Calif.) using Softmax® Pro 3.0 software (Molecular Devices Corporation). Each experiment was performed in three replicates and was repeated twice.

This assay was performed on soy products processed seven different ways. Example A was soybeans ground into powder (Sunlight Foods Corporation, Taipei County, Taiwan, R.O.C.). Example B was soybean powder of Example A exposed to about 8-15 kGy of gamma irradiation. Example C was soybean powder in which the oil in the soybean powder was removed by extraction (Soyafluff® 200W from Central Soya Company, Inc., Fort Weyne, Ind.). Example D was soymilk powder made with dehulled soybeans and water that was subsequently filtered and heated and spray dried (Devansoy Farms, Carroll, Iowa) and exposed to between about 7-9 kGy of gamma irradiation. Example E was soymilk powder obtained by mixing soy beans and water, heating the mixture overnight, and adding 1,3-butylene glycol to the mixture (Flavosterone SB from Ichimaru Pharcos Co., Ltd, Gifu Japan). Example F was soymilk powder obtained by mixing soy beans and water, heating the mixture overnight, and subsequently adding ethanol to the mixture (Flavosterone SE from Ichimaru Pharcos Co., Ltd, Gifu Japan). Example G was an extract of soy proteins (Vegeseryl HGP LS 8572 from Laboratories Serobiologiques S.A., Pulnoy, France). These soy products were compared to Soy Trypsin Inhibitor (STI) (Sigma).

The percent inhibition of trypsin cleavage of the substrate by the different soy preparations was calculated using Microsoft Excel™ and is reported in Table 1.

TABLE 1

| Tested | | % Inhibition of |
|---|---|---|
| Product | Concentration | Trypsin |
| STI | 0.01 | 43.0 |
| STI | 0.1 | 76.1 |
| Example A | 0.01 | 32.8 |
| Example A | 0.1 | 67.1 |
| Example B | 0.01 | 31.5 |
| Example B | 0.1 | 67.2 |
| Example C | 0.01 | 22.7 |
| Example C | 0.1 | 36.2 |
| Example D | 0.01 | 8.92 |
| Example D | 0.1 | 17.4 |
| Example E | 0.01 | 7.83 |
| Example E | 0.1 | 10.8 |
| Example F | 0.01 | 4.87 |
| Example F | 0.1 | 5.99 |
| Example G | 0.1 | 6.85 |

As shown in Table 1, STI can inhibit trypsin-induced cleavage in a dose response manner. Example A, which is soybean powder, also significantly inhibited trypsin activity. Further gamma irradiation of the soybean powder (i.e., Example B), while reducing the microbial content of the soybean powder, unexpectedly did not significantly impact the trypsin inhibition activity of the soybean powder. The heat and/or extraction processing of Examples C-G, however, did significantly reduce the trypsin inhibitory activity of the soybean powder.

Topical Compositions

The topical compositions useful in the present invention involve formulations suitable for topical application to skin.

The composition preferably contains a non-denatured soy product and a cosmetically-acceptable topical carrier. The cosmetically-acceptable topical carrier is preferably from about 0.1% to about 99.99%, by weight, of the composition (more preferably, from about 80% to about 95%, by weight, of the composition). Topical compositions useful in the subject invention may be formulated as a solution comprising an emollient, or emollients. Such compositions preferably contain from about 2% to about 50% of an emollient(s). A lotion can be made from such a solution. Lotions typically comprise from about 1% to about 20% (e.g., from about 5% to about 10%) of an emollient(s) and from about 50% to about 90% (more preferably, from about 60% to about 80%) of water. A cream typically comprises from about 5% to about 50% (more preferably, from about 10% to about 20%) of an emollient(s) and from about 45% to about 85% (more preferably, from about 50% to about 75%) of water. The topical compositions useful in the present invention formulated as emulsions. If the carrier is an emulsion, from about 1% to about 10% (more preferably, from about 2% to about 5%) of the carrier is made up of at least one emulsifier. Emulsifiers may be nonionic, anionic or cationic.

The non-denatured soy product content of the compositions useful in the methods of this invention is preferably from about 0.001% to about 99.9% by weight of the composition. More preferably, the content should be from about 0.01% to about 50%, and most preferably from about 0.5% to about 50%. The compositions of this invention may be created in powder form, in which case the soy product content may be present in amounts of from about 1 to about 99% of the composition. Soy product can be soymilk made from soymilk powder or soybean or soymilk powder or soybean powder, as set forth in the referred-to patent applications.

Additional cosmetic actives include anti-aging agents, anti-irritant agents, anti-cellulite agents, lightening agents, darkening agents such as self-tanning agents, anti-acne agents, shine control agents, anti-microbial agents, anti-bacteria agents, anti-inflammatory agents, anti-mycotic agents, anti-parasite agents, external analgesics, sunscreens, photoprotectors, antioxidants, keratolytic agents, moisturizers, nutrients, vitamins, energy enhancers, anti-perspiration agents, skin calming agents, skin protectant agents,skin healing agents, skin soothing agents, astringents, deodorants, hair removers, firming agents, anti-callous agents, and agents for hair, nail, and/or skin conditioning. AHA,antiseptic agents, essential oils, proteins, silicones and the like.

Surprisingly, the compositions of this invention, when applied to the skin one or more times, act to “balance” both the moisture level and sebum level of the skin. The same application of composition of this invention both confers moisture to dry areas of the skin and decreases sebum production in oily areas of the skin. Thus, it selectively restores moisture where needed and reduces the level of oiliness in areas where such reduction is desired.

The following example illustrates, but does not limit the scope of the compositions and methods of this invention.

### EXAMPLE 3

Total Soy Lotion for Skin Balancing

A clinical study was designed to assess the potential of a novel Total Soy lotion to balance combination skin and to affect tactile properties. A five-week, half-face, double blind placebo-controlled clinical study was conducted on twenty-three female subjects ages 20-35 years with combination skin (Fitzpatrick type I-II). “Combination skin”: was defined as facial skin having at least one oily area and one dry area on each of the half face. The oily and dry areas were determined by sebumeter reading at >200 g/cm² and <66 g/cm², respectively. Subjects applied soy lotion or placebo on the designated side of the face daily for 5 weeks. Measurements were taken at baseline, weeks 1, 3 and 5 on the forehead, cheek and chin. Scaling, moisturization, oiliness and smoothness were evaluated by instrumental measurements and digital photography. The results indicated this Total Soy preparation significantly reduced sebum in oily patches (p<0.05 for chin areas) and enhanced moisturization for dry patches as compared to placebo (p<0.05). Significant changes were not observed in single tape stripping for scaling or digital photography. Subject self-assessments demonstrated enhanced skin smoothness and oil reduction, which correlated with the instrumental results. Approximately 70% of the subjects noted improvement on the soy treated side while only 17% on placebo treated side noted improvement in overall tone and texture. In summary, this clinical study demonstrated that the Total Soy preparation balanced tactile properties of combination skin by regulating skin oily and dry areas, i.e., selectively reduced oiliness of oily patches and enhanced moisturization of dry patches.

Parameters

Dryness/scaling

Moisturization

Oiliness

Smoothness

### Methods

5-Week Half-Face, Double Blind Placebo-Controlled Study Inclusion Criteria

Adult females, age: 20-35 years

Fitzpatrick Skin Type I & II (n=23)

Pre-screen with sebum analysis

Product Application

Pre-condition—initial wash with liquid cleanser for 3 days

Total Soy lotion vs placebo lotion using split-face design

Daily PM application of Total Soy lotion and placebo for 5 weeks

Total Soy Lotion contained the following ingredients:

| Ingredient | W/W Percentage |
|---|---|
| Deionized Water | 72.43% |
| Chelators | 1.2% |
| Preservatives | 1.6% |
| Glycerine | 3% |
| Solvent | 5% |
| Antioxidant | 0.1% |
| Emulsifiers | 4.9% |
| Silicone skin conditioners | 8.15% |
| Thickener | 1% |
| Soymilk powder | 2.5% |
| Soy isoflavone extract (65%) | 0.12% |

Assessment

The following instrumental measurements were taken at 0, 1, 3 and 5 weeks:

Skin hydration by Novameter®

Sebum analysis by Sebumeter®

All study subjects also maintained a diary

Methods of Measurement

Sebum Measurement by Sebumeter:

The technique chosen for sebum measurement is the Courage and Kzahaka Sebumeter Model SM810. The principle of the Sebumeter involves the use of photometry with special opaque plastic film in a hand-held cartridge. This film becomes more transparent as more sebum is collected or deposited onto it. For sebum measurements, the cartridge is placed onto the test area for 30 seconds to absorb the surface sebum. The film is measured for transparency with a high sebum content causing a higher readout. The resulting Sebumeter value is calibrated for units of μg sebum/cm².

Moisturization Measurement by Nova DPM 9003:

It has been recognized that water plays a fundamental role in controlling and modifying the physical properties of stratum corneum. Skin electrical properties are expressed as its electric potential as its resistance to electric current flow. This resistance is determined by applying an external voltage across two surface sites and finding the transient and/or steady state patterns of transcutaneous electrical current flow between these sites. The measurements can be resistance, conductance or impedance. There is evidence that these electrical signals might correlate with skin surface hydration. Commercially available measurement devices using electrical methods allow quantitative evaluation of the skin function and moisturization. Such instruments include the Skicon, the Corneometer and the Dermal Phase Meter (DPM). In this study, Nova DPM 9003 delivers impedance-based capacitance readings by integrating selected measurements at varying frequencies of the applied alternating current. The higher the capacitance reading or the DPM reading is, the higher the skin hydration is.

### Criteria of Combination Skin

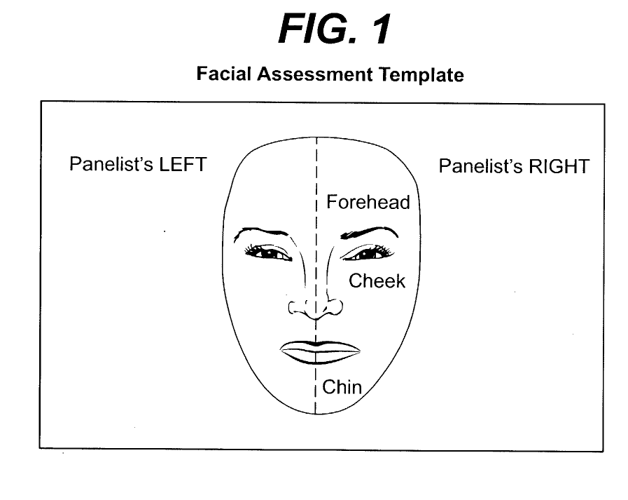

Subjects were categorized as having “combination skin” if their faces had at least one oily area (sebum level>200 mg/cm²) and one dry area (sebum level<66 mg/cm²) on each half face. The Facial Assessment Template set forth in FIG. 1 demonstrates how evaluation was identified with respect to each subject.

FIG. 1 demonstrates different evaluation sites on subject's face. The facial mask was designed and used for the evaluation.

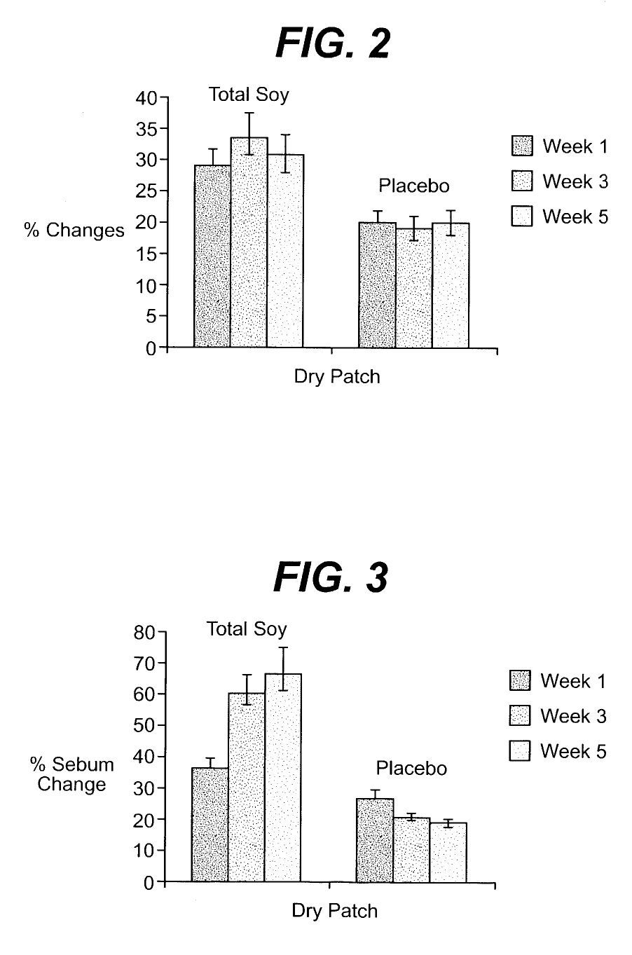

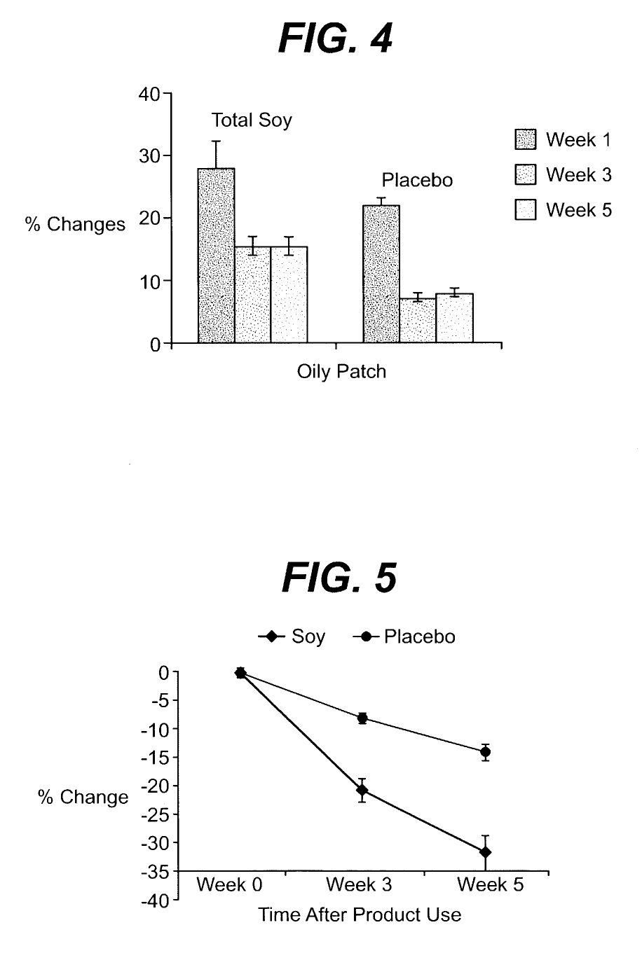

The Total Soy lotion treated sites had increased moisturization compared with placebo for dry patches (p<0.05) as set forth in FIG. 2. The evaluations were made 12-24 hours after topical applications. FIG. 3 demonstrates that Total Soy lotion balanced sebum levels of dry skin patches vs placebo (mean±SD; p<0.05). FIG. 4 above demonstrates that Total Soy lotion treated sites balanced oily skin patches (mean±SD). The Total Soy lotion selectively decreased sebum levels of oily patches for chin area vs. placebo (p<0.05), as set forth in FIG. 5.

TABLE 2
Subject Evaluation

| | PLACEBO | TOTAL SOY |
|---|---|---|
| Breakout/acne | 35.3% | 5.9% |
| Improvement in skin | 5.9% | 35.3% |
| smoothness/softness | | |
| Noticeable reduction in | 17.6% | 58.8% |
| skin oiliness/laugh | | |
| line/redness | | |

At the end of study (week 5), for those reported subject evaluations, Total Soy was rated more favorably than placebo. Approximately 60% of the subjects noted improvement on the soy treated side while only 17% on placebo treated side noted improvement in overall tone and texture. Such improvements also included the fine wrinkle line reduction. Clearly, “total soy” also provides additional skin benefits in the areas of even tone and texture, softness, and wrinkle reduction as set forth in the abovementioned copending patent applications, Such compositions are described in copending U.S. patent applications Ser. No. 09/110,409, filed Jul. 6, 1998, Ser. No. 09/206,249, filed Dec. 7, 1998, Ser. No. 09/361,429, filed Jul. 27, 1999, Ser. No. 09/621,565, filed Jul. 21, 2000 and Ser. No. 09/698,454, filed Oct. 27, 2000, which are hereby incorporated herein by reference.

Among the placebo testing subjects, a significant portion (35.3%) reported the acne or breakout events than the total soy subjects (5.9%) (See Table 2 above). These results are significant. As one would expect, the oily skin or excess skin sebum is a necessary condition for acne-prone skin. The reduction of excessive sebum on the oily patches resulted in the reduction of acne breakout. Total soy can be used to treat the acne prone skin to reduce the likelihood of acne.

The compositions of this invention, Total Soy contains a broad-spectrum of non-denatured active components that were shown to provide a number of skin care benefits. In this study on combination facial skin, we learn that:

Total Soy lotion moisturized dry patches

Total Soy lotion selectively reduced sebum levels of facial skin oily patches

Total Soy lotion clinically evened out dry/oily patches in combination facial skin

Total Soy effectively smoothed and softened facial skin texture

Total soy's dual actions on sebum reduction and moisturizing are believed to be due to the fact that it comprises up to 40-50% protein, 20-30% of triglyericdes, made up of long chain fatty acids (C16-C20 and C22), of which, a large proportion is polyunsaturated fatty acids and 20-30% carbohydrates as well as minor components such as soy isoflavones, phytosterols, vitamins and minerals. These components can contribute to the reduction of excess sebum for oily or greasy skin areas and at the same time to impart the hydrating effect on the dry areas.

What is claimed is:

1. A composition for balancing the appearance of combination skin comprising a legume product.

2. A composition according to claim 1, wherein said legume is soy.

3. A composition of claim 2, wherein said soy is total soy.

4. A method of balancing the appearance of combination skin comprising applying a legume product to skin having both oily and dry components.

5. A method according to claim 4 wherein said legume product is a soy product.

6. A method according to claim 5 wherein said soy product is total soy.

7. A method of treating skin having at least one area wherein the initial sebum level of said skin is at least about 200 μg/cm² and at least one area wherein the initial sebum level of said skin is less than about 66 μg/cm² comprising applying to said skin a composition comprising total soy at least once per day for a period of time sufficient to achieve a balanced appearance, reduce the sebum level in the at least one area having an initial sebum level of at least about 200 μg/cm² and increasing the sebum level in the at least one area wherein the initial sebum level of said skin is less than about 66 μg/cm².

8. A method according to claim 9 further comprising the step of applying said total soy composition at least twice per day.

9. A method according to claim 9 wherein said application of total soy is performed over the period of at least three weeks.

10. A method of treating skin having at least one area wherein the initial sebum level of said skin is at least about 150 μg/cm² and at least one area wherein the initial sebum level of said skin is less than about 100 μg/cm² comprising applying to said skin a composition comprising total soy at least once per day for a period of time sufficient to achieve a balanced appearance, reduce the sebum level in the at least one area having an initial sebum level of at least about 150 μg/cm² and increasing the sebum level in the at least one area wherein the initial sebum level of said skin is less than about 100 μg/cm².
